SEQUENCE LISTING (44 sequences)

SEQ ID NO: 1 - DNA, 27 bp, Artificial Sequence
  27-mer forward primer
cgattgcccatgggtaaaggagaactt

SEQ ID NO: 2 - DNA, 32 bp, Artificial Sequence
  32-mer reverse primer
tgcaggagctcctagatagatctgtatagttc

SEQ ID NO: 3 - DNA, 33 bp, Artificial Sequence
  33-mer forward primer
gcgggctctagaatgcccgatcgtgctcaagtt

SEQ ID NO: 4 - DNA, 35 bp, Artificial Sequence
  35-mer reverse primer
cttgagcgggagcaggctcccatgggagctcctgc

SEQ ID NO: 5 - DNA, 35 bp, Artificial Sequence
  35-mer forward primer
gcggcctcgagatgggttctatgatatcctcttcc

SEQ ID NO: 6 - DNA, 45 bp, Artificial Sequence
  45-mer reverse primer
atgcaggagctccatgggtctagagcactttactcttccaccatt

SEQ ID NO: 7 - DNA, 35 bp, Artificial Sequence
  35-mer forward primer
gcggcaagcttatgggttctatgatatcctcttcc

SEQ ID NO: 8 - DNA, 45 bp, Artificial Sequence; identical to SEQ ID NO: 6
  45-mer reverse primer

SEQ ID NO: 9 - DNA, 29 bp, Artificial Sequence
  29-mer forward primer
gcggctctagaatgaagctactgtcttct

SEQ ID NO: 10 - DNA, 35 bp, Artificial Sequence
  35-mer reverse primer
gcaggagctcccatgggaatcgatacagtcaactg

SEQ ID NO: 11 - DNA, 729 bp, Artificial Sequence
  5′-NcoI-gfp-SstI-3′
cccatgggtaaaggagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccctggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacagatctatctaggagctc

SEQ ID NO: 12 - DNA, 636 bp, Artificial Sequence
  5′-XbaI-VirD2-C-NcoI-SstI-3′
tctagaatgcccgatcgtgctcaagttatcattcgcattgtgccgggaggtggcaccaagacccttcaacaaattatcaatcagttggagtatctatcccggaagggcaggctggagctgcagcgttcagcccgacatctcgatattcccctgccaccggatcaaatccacgtacttgcccgaagctgggttcaagagactggaacttatgacgaaagtcagccagacgaggaaaggcaacaggagttgaccacccatattattgtaagcttccccgccggtacaagccaggtagcggcttatgcggcgagccgggagtgggcagccgagatgtttgggtcaggcgcaggggggggccgatacaactatcttacggccttccacatcgatcgcgaccacccacatctgcatgtcgtcgtcaatcggcgcgaacttttaggacacggctggctgaagatatctcggcgccatccccaactgaattacgacgccctgcgcataaagatggccgagatttcacttcgtcatggcattgccctcgatgcgagccgacgagcagaacgtggcatcaccgagcggccgatcacttatgcccaatatcggcgccttgagcgggagcaggctccatgggagctc

SEQ ID NO: 13 - DNA, 196 bp, Artificial Sequence
  5′-XhoI-cTP-XbaI-C-NcoI-SstI-3′
ctcgagatgggttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgctctagacccatgggagctc

SEQ ID NO: 14 - DNA, 196 bp, Artificial Sequence
  5′-HindIII-cTP-XbaI-C-NcoI-SstI-3′
aagcttatgggttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgctctagacccatgggagctc

SEQ ID NO: 15 - DNA, 463 bp, Artificial Sequence
  5′-G4dbd-C-NcoI-SstI-3′
tctagaatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgattcccatgggagctc

SEQ ID NO: 16 - DNA, 1356 bp, Artificial Sequence
  5′-HindIII- cTP-XbaI-G4dbd-NcoI-GFP-SstI-3′
aagcttatgggttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgctctagaatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgattcccatgggtaaaggagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccctggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacagatctatctaggagctc

SEQ ID NO: 17 - DNA, 1530 bp, Artificial Sequence
  5′-XhoI-cTP-XbaI-VirD2-NcoI-GFP-SstI-3′
ctcgagatgggttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgctctagaatgcccgatcgtgctcaagttatcattcgcattgtgccgggaggtggcaccaagacccttcaacaaattatcaatcagttggagtatctatcccggaagggcaggctggagctgcagcgttcagcccgacatctcgatattcccctgccaccggatcaaatccacgtacttgcccgaagctgggttcaagagactggaacttatgacgaaagtcagccagacgaggaaaggcaacaggagttgaccacccatattattgtaagcttccccgccggtacaagccaggtagcggcttatgcggcgagccgggagtgggcagccgagatgtttgggtcaggcgcaggggggggccgatacaactatcttacggccttccacatcgatcgcgaccacccacatctgcatgtcgtcgtcaatcggcgcgaacttttaggacacggctggctgaagatatctcggcgccatccccaactgaattacgacgccctgcgcataaagatggccgagatttcacttcgtcatggcattgccctcgatgcgagccgacgagcagaacgtggcatcaccgagcggccgatcacttatgcccaatatcggcgccttgagcgggagcaggctcccatgggtaaaggagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccctggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacagatctatctaggagctc

SEQ ID NO: 18 - DNA, 39 bp, Artificial Sequence
  39-mer forward primer
gcggaattcctgcagggatccgctcccccgccgtcgttc

SEQ ID NO: 19 - DNA, 27 bp, Artificial Sequence
  27-mer reverse primer
atgaagcttaaatccctccctacaact

SEQ ID NO: 20 - DNA, 21 bp, Artificial Sequence
  21-mer forward primer
gcggcttgcatgcctgcaggt

SEQ ID NO: 21 - DNA, 27 bp, Artificial Sequence
  27-mer reverse primer
atgggatcctctagcgtctccgctcgg

SEQ ID NO: 22 - DNA, 27 bp, Artificial Sequence
  27-mer forward primer
gcgtctagagatcctggcctagtctat

SEQ ID NO: 23 - DNA, 27 bp, Artificial Sequence
  27-mer reverse primer
atgatcgattcgaatatagctcttctt

SEQ ID NO: 24 - DNA, 159 bp, Artificial Sequence
  5′EcoR1-PstI-BamHI-Prnn promoter-HindIII-3′
gaattcctgcagggatccgctcccccgccgtcgttcaatgagaatggataagaggctcgtgggattgacgtgagggggcagggatggctatatttctgggagcgaactccgggcgaatacgaagcgcttggatacagttgtagggagggatttaagctt

SEQ ID NO: 25 - DNA, 105 bp, Artificial Sequence
  5′-PstI-5UAS-BamHI
ctgcagcggagtactgtcctccgagcggagtactgtcctccgagcggagtactgtcctccgagcggagtactgtcctccgagcggagtactgtcctccgggatcc

SEQ ID NO: 26 - DNA, 405 bp, Artificial Sequence
  5′-XbaI-psbA3′-ClaI-3′
tctagagatcctggcctagtctataggaggttttgaaaagaaaggagcaataatcattttcttgttctatcaagagggtgctattgctcctttctttttttctttttatttatttactagtattttacttacatagacttttttgtttacattatagaaaaagaaggagaggttattttcttgcatttattcatgattgagtattctattttgattttgtatttgtttaaattgtgaaatagaacttgtttctcttcttgctaatgttactatatctttttgattttttttttccaaaaaaaaaatcaaattttgacttcttcttatctcttatctttgaatatctcttatctttgaaataataatatcattgaaataagaaagaagagctatattcgaatcgat

SEQ ID NO: 27 - DNA, 50 bp, Artificial Sequence
  5′-XhoI-5′amv-NcoI-3′
cctcgagcggatctttttatttttaattttctttcaaatacttccatggg

SEQ ID NO: 28 - DNA, 635 bp, Artificial Sequence
  5′-NcoI-cTP-g4dbd-SalI-SstI-3′
ccatgggttctatgatttcttcttctgctgtgactactgtgtctagggcttctaggggacaatctgctgctgttgctccatttggaggacttaagtctatgactggattcccagtgaagaaggtgaacactgatattacttcaattacttctaacggtggaagggtgaagtgtatgaagttgctttcttctattgagcaggcttgcgatatctgccgtcttaagaaacttaagtgctctaaggaaaagccaaagtgcgctaagtgccttaagaataactgggagtgccgttactctccaaagactaagaggtctccacttactagggctcaccttactgaagttgagtctaggcttgaaaggcttgagcagcttttccttcttattttcccaagggaggatcttgatatgattcttaagatggattctcttcaggatattaaggctcttcttactggacttttcgttcaggataacgtgaacaaggatgctgtgactgataggcttgcttctgtggagactgatatgccacttactcttaggcagcacaggatttctgctacttcatcttctgaggagtcatctaacaagggacagaggcagcttactgtttctattgggtcgacttgagagctc

SEQ ID NO: 29 - DNA, 644 bp, Artificial Sequence
  EcoRI-M24 promoter-HindIII
gaattcgtcaacttcgtccacagacatcaacatcttatcgtcctttgaagataagataataatgttgaagataagagtgggagccaccactaaaacattgctttgtcaaaagctaaaaaagatgatgcccgacagccacttgtgtgaagcatgtgaagccggtccctccactaagaaaattagtgaagcatcttccagtggtccctccactcacagctcaatcagtgagcaacaggacgaaggaaatgacgtaagccatgacgtctaatccccccaacttcgtccacagacatcaacatcttatcgtcctttgaagataagataataatgttgaagataagagtgggagccaccactaaaacattgctttgtcaaaagctaaaaaagatgatgcccgacagccacttgtgtgaagcatgtgaagccggtccctccactaagaaaattagtgaagcatcttccagtggtccctccactcacagctcaatcagtgagcaacaggacgaaggaaatgacgtaagccatgacgtctaatcccacaagaatttccttatataaggaacacaaatcagaaggaagagatcaatcgaaatcaaaatcggaatcgaaatcaaaatcggaatcgaaatctctcatctaagctt

SEQ ID NO: 30 - DNA, 20 bp, Artificial Sequence
  20-mer antisense sequence
aatgataacttgagcacgat

SEQ ID NO: 31 - DNA, 20 bp, Artificial Sequence
  20 mer sense sequence
aacttatgacgaaagtcagc

SEQ ID NO: 32 - DNA, 20 bp, Artificial Sequence
  20-mer antisense sequence
gagatatcttcagccagccg

SEQ ID NO: 33 - DNA, 20 bp, Artificial Sequence
  20-mer sense sequence
gcgagccgacgagcagaacg

SEQ ID NO: 34 - DNA, 20 bp, Artificial Sequence
  20-mer antisense sequence
cttgacttcagcacgtgtct

SEQ ID NO: 35 - DNA, 20 bp, Artificial Sequence
  20-mer antisense sequence
gaaaaatcagtagaaatagc

SEQ ID NO: 36 - DNA, 21 bp, Artificial Sequence
  21-mer sense sequence
aaggctagaaagactggaaca

SEQ ID NO: 37 - DNA, 20 bp, Artificial Sequence
  20-mer sense sequence
ggttctatgatatcctcttc

SEQ ID NO: 38 - DNA, 20 bp, Artificial Sequence
  20-mer anti sense sequence
tctagagcactttactcttc

SEQ ID NO: 39 - DNA, 20 bp, Artificial Sequence
  20-mer sense sequence
atgcccgatcgtgctcaagt

SEQ ID NO: 40 - DNA, 20 bp, Artificial Sequence
  20-mer antisense sequence
ccctgcctgctcccgctcaa

SEQ ID NO: 41 - DNA, 20 bp, Artificial Sequence
  20-mer sense sequence
atgggtaaaggagaactttt

SEQ ID NO: 42 - DNA, 20 bp, Artificial Sequence
  20-mer antisense sequence
ctagatagatctgtatagtt

SEQ ID NO: 43 - DNA, 20 bp, Artificial Sequence
  20-mer sense sequence
atgaagctactgtcttctat

SEQ ID NO: 44 - DNA, 20 bp, Artificial Sequence
  20-mer antisense sequence
gggaatcgatacagtcaact

(19) United States
(12) Patent Application Publication
(10) Pub. No.: US 2010/0319094 A1
(43) Pub. Date: Dec. 16, 2010

(54) METHODS AND COMPOSITIONS FOR GENETIC TRANSFORMATION OF PLANT CHLOROPLASTS

(76) Inventors: INDU BHUSHAN MAITI, LEXINGTON, KY (US); DEANE L. FALCONE, WESTFORD, MA (US); SUMITA RAHA, CHICAGO, IL (US); H. MAELOR DAVIES, LEXINGTON, KY (US)

Correspondence Address: KING & SCHICKLI, PLLC, 247 NORTH BROADWAY, LEXINGTON, KY, 40507, US

(21) Appl. No.: 12/483,481

(22) Filed: Jun. 12, 2009

(51) Int. Cl. A01H 5/00; C12N 15/82; C12N 15/00; C12N 5/04

(52) U.S. Cl. 800/317.3; 800/298; 435/468; 435/320.1; 435/419

(57) ABSTRACT

A method for plastid transformation of a plant cell includes providing a delivery construct for transferring a passenger construct into a plant cell plastid for expression of a foreign DNA of interest therein. The delivery construct includes a nucleotide sequence under the control of a suitable promoter, for encoding at least one fusion protein which is a chloroplast transit peptide and a DNA binding domain. The passenger construct includes a nucleotide sequence encoding at least one foreign protein of interest or fragment thereof to be expressed in the plant cell, under the control of a plastid-specific promoter.

### TECHNICAL FIELD

The present invention relates to transformation of plant cells for expression of desired proteins or peptides, including expression in multi-cellular plants. In particular, the invention relates to novel methods and compositions for plastid transformation of plant cells, and for expression of foreign DNA of interest in plant cells, including in multi-cellular plants.

### BACKGROUND OF THE INVENTION

In higher plants, the chloroplast/plastid is the site of photosynthesis. The plastid DNA (size 120-160 kb) encodes genes involved in photosynthesis and in plastid maintenance. In the stroma, chloroplasts contain the entire enzymatic machinery for carbohydrate biosynthesis. Chloroplasts also serve as compartments for biosynthesis of complex molecules including without limitation amino acids, fatty acids, vitamins and pigments (Boyer et al, 1989). The plant cell nucleus encodes most of the genes involved in chloroplast functions, and related enzymes synthesized on cytoplasmic ribosomes are subsequently transported to plastids. This process of gene expression and accumulation of products in nucleus and chloroplasts, respectively, is well coordinated (Gruissem, 1989, Zurawski and Clegg, 1987). The chloroplast genome is highly conserved and organized (Palmer 1991, Raubeson and Jansen 2005), in general, this is composed of a single circular chromosome with two copies of an inverted repeat (IR) separated by the large and small single copy regions: LSC and SSC, respectively, (Jansen et al., 2005).

An important goal of plant biotechnology is high-level production of commercially and pharmaceutically important proteins/peptides in plants. Transgenic plants are being developed for basic research to study the gene function and regulation, as well as to improve agronomically important crop plants (Benfey and Chua, 1989; Weising, et al., 1988, Maiti and Hunt, 1992, Wagner 1992). Transformation technology for nuclear genomes is very routinely applied. On the other hand, transformation technology for the genomes of cytoplasmic organelles such as plastids and mitochondria remains a nascent technology, and is not routinely practiced.

The chloroplasts of higher plants accumulate individual components of the photosynthetic machinery as a relatively large fraction of total cellular protein. The best example is the enzyme ribulose-1,5-bisphosphate carboxylase-oxygenase (Rubisco), involved in CO₂ fixation, which can make up more than 50% of the total leaf protein. Because of the potentially attainable high protein levels, there is significant interest in exploring chloroplasts as an alternative system for protein expression. Current methods for chloroplast transformation include biolistics, polyethylene glycol (PEG)-mediated transformation & developing chloroplast-specific vectors to facilitate the incorporation of the transgenes into the chloroplast genome.

Transformation/genetic engineering of plastid genomes generally occurs by a recombination process of the transforming DNA through the plastid inverted repeats. Exogenous DNA integrates into plastid genomes by homologous recombination through the plastid inverted repeat sequences (Palmer 1985). A variety of methods for introduction of exogenous DNA into the plastid genome have been attempted, including calcium phosphate co-precipitation (Krens et al., 1982), electroporation (Forman et al, 1986), polyethylene glycol treatment (Negrutiu, et al., 1987), incubation in the presence of EDTA (Daniell and McFadden 1987), Agrobacterium-mediated transformation (Weising et al, 1988) and transformation by high-velocity microprojectiles (Klein et al., 1988, Daniell et al., 1990).

Despite reported successes from a few laboratories in the world, chloroplast transformation is not still a routine procedure, as it is time-consuming and cumbersome compared to conventional nuclear transformation. However, chloroplast transformation technology is in theory a promising tool in biotechnology and has the potential to solve some of the problems associated with traditional plant genetic engineering. Chloroplast genetic engineering provides a number of potential advantages, including high-level expression of a foreign gene (De Cosa et al., 2001), expression of multiple genes in a single cell (Shinozaki et al., 1986), transgene containment (Polans et al., 1990; Daniell et al., 1998), and minimizing gene silencing as well as position effect (Lee et al., 2006).

For these and other advantages, an effective, efficient and routine method for plastid transformation is a desired goal in plant biotechnology. Due to the potential advantages of chloroplast transformation technology compared to nuclear genome transformation, there has been a recent surge in sequencing the whole chloroplast genome of various organisms. Complete chloroplast genomic sequences of a number of important plant species including tobacco, tomato, soybean, cucumber, cotton and Eucalyptus, have been reported (Jansen et al., 2005; Lee et al., 2006). Presently, seventy chloroplast genomes representing sixty-four organisms are available.

### SUMMARY OF THE INVENTION

To solve the aforementioned and other problems, a novel strategy is described herein for alternative transformation of chloroplasts (plastids), demonstrating plastid expression of foreign DNA in multicellular plants. In one aspect, the presently described method includes providing a delivery construct including a nucleotide sequence encoding at least one fusion protein comprising a chloroplast transit peptide and a DNA binding domain. Expression of the at least one fusion protein nucleotide sequence is under the control of a promoter. A separate passenger construct is provided, including a nucleotide sequence encoding at least one foreign protein of interest or fragment thereof to be expressed in the plant cell. The delivery construct and the passenger construct are co-introduced into a plant cell under conditions whereby the delivery construct transfers the passenger construct into the plant cell plastid and the at least one foreign protein or fragment thereof is expressed in the plant cell.

In another aspect, the present invention provides a deli very system for plastid transformation of a plant cell, including a delivery construct and a passenger construct as described above. The passenger construct includes a plastic-specific promoter, a nucleotide sequence encoding at least one foreign protein of interest or fragment thereof to be expressed in the plant cell, and a terminator sequence. Still further, the present invention provides plant cells and multicellular plants plastid-transformed with the delivery system described herein.

These and other embodiments, aspects, advantages, and features will be set forth in the description which follows, and in part will become apparent to those of ordinary skill in the art by reference to the following description of the invention and referenced drawings or by practice of the invention. The aspects, advantages, and features of the invention are realized and attained by means of the instrumentalities, procedures, and combinations particularly pointed out in the appended claims. Various patent and non-patent references are discussed herein. Unless otherwise indicated, any such references are incorporated in their entirety into the present disclosure specifically by reference.

### BRIEF DESCRIPTION OF THE DRAWINGS

The accompanying drawings, incorporated herein and forming a part of the specification, illustrate several aspects of the present invention and together with the description serve to explain certain principles of the invention. In the drawings:

FIG. 1 presents a schematic map of an empty plant expression vector pKM24 with a modified full-length transcript promoter (M24) from the Mirabilis mosaic virus (MMV; Dey and Maiti, 1999a,b), showing the left T-DNA border (LT), the right T-DNA border (RT), the M24 promoter, multiple cloning sites (MCS, 5′-HindIII-BamHI-XhoI-PstI-SacI-XbaI-3′, unique sites are shown in bold), the 3′-terminator sequences (Terminators) of the ribulose1,5-bisphosphate carboxylase rbcSE9, (rbcS3′) and nopaline synthase (3′ Nos) genes, and the neomycin phosphotransferase II marker gene (Kan^{R}) directed by nopaline synthase promoter (NosP);

FIG. 2 presents a schematic map of pKM24-GUS, a positive control vector including a chimeric GUS reporter gene directed by the M24 promoter, showing also the position of the EcoR1, HindIII, XhoI, XbaI, SstI and ClaI restriction sites used to assemble the expression vectors;

FIG. 3 presents a schematic map of pKM24-GFP, a positive control vector including a chimeric GFP reporter gene directed by the M24 promoter;

FIG. 4 presents a schematic map of pKM24-LUC, a positive control vector including a chimeric LUC reporter gene directed by the M24 promoter;

FIG. 5 presents a schematic map of pKPrnn-GUS, a control vector including a chimeric GUS reporter gene directed by the Prnn promoter;

FIG. 6 presents a schematic map of pKPrnn-GFP, a control vector including a chimeric GUS reporter gene directed by the Prnn promoter;

FIG. 7 presents a schematic map of pKPrnn-LUC, a control vector including a chimeric LUC reporter gene directed by the Prnn promoter;

FIG. 8 presents a schematic map of pKM24cTP-GFP, a chloroplast targeting positive control vector, including a chimeric GFP gene fused with the chloroplast targeting peptide (cTP), directed by the M24 promoter;

FIG. 9 presents a schematic map of the test chloroplast targeting delivery construct pKM24-cTP-G4dbd-GFP containing chimeric GFP gene fused in frame with Gal4 DNA binding domain (G4dbd) and cTP, directed by the M24 promoter;

FIG. 10 presents a schematic map of pKM24cTP-G4dbd, a delivery construct containing a chimeric G4dbd fused in frame with cTP, directed by the M24 promoter;

FIG. 11 presents a schematic map of the passenger construct pK5uasPrnn-GUS containing a chimeric GUS reporter gene directed by the modified Prnn promoter fused with 5XUAS, and also showing the 3′-terminator sequence psbA3;

FIG. 12 presents a schematic map of the passenger construct pK5uasPrnn-GFP containing a chimeric GFP reporter gene directed by the modified Prnn promoter fused with 5XUAS;

FIG. 13 presents a schematic map of the passenger construct pK5uasPrnn-LUC containing a chimeric LUC reporter gene directed by the modified Prnn promoter fused with 5XUAS;

FIG. 14 presents laser scanning confocal microscope images showing a transient expression analysis of pKM24-GFP (positive control; FIG. 3) after biolistic transformation of Nicotiana tabacum leaf tissue using a microprojectile gene gun, with expression of the GFP reporter shown in the nucleus (yellow arrow) and throughout the subsidiary cell, and chloroplasts (white arrow) showing red color due to chlorophyll autofluorescence;

FIG. 15 presents laser scanning confocal microscope images showing a transient expression analysis of pKM24cTP-GFP (delivery construct; FIG. 8) in N. tabacum leaf tissue after biolistic transformation, demonstrating exclusive localization of the reporter GFP in the chloroplast (panel A, white arrow), with panel B showing chlorophyll autofluorescence and Panel C providing an overlay image showing both GFP fluorescence and chlorophyll autofluorescence expression in the nucleus (yellow arrow) and throughout the subsidiary cell [chloroplasts (white arrow) show red color due to chlorophyll autofluorescence];

FIG. 16 presents laser scanning con focal microscope images showing a transient expression analysis of pKM24cTP-G4dbd-GFP (test targeting construct; FIG. 9) in N. tabacum leaf tissue after biolistic transformation, demonstrating localization of the reporter GFP in the nucleus (panel A, yellow arrow) and in a few chloroplasts (panel A, white arrow), with panel B showing a red color due to chlorophyll autofluorescence and panel C showing an overlay image of the combined images of both GFP and chlorophyll fluorescence expression in the nucleus (yellow arrow) and chloroplast (white arrow);

FIG. 17 presents laser scanning confocal microscope images showing a transient expression analysis of pKM24cTP-G4dbd-GFP (test targeting construct; FIG. 9) in N. tabacum leaf tissue after biolistic transformation, demonstrating localization of the reporter GFP in a few chloroplasts (panel A, white arrow), with panel B showing a red color due to chlorophyll autofluorescence and panel C showing an overlay image of the combined images of both GFP and chlorophyll fluorescence expression in the nucleus (yellow arrow) and chloroplast (white arrow);

FIG. 18 presents an integration analysis of pKM24cTP-G4dbd-GFP (test targeting construct; FIG. 9) in transgenic tobacco by PCR amplification of G4dbd in genomic DNA from independent lines, showing the expected bands from independent tobacco transformed lines (individual line number indicated at the top, lane 1 to 13), DNA size Marker (lane M), positive control (lane Pc);

FIG. 19 presents an expression analysis of pKM24cTP-G4dbd-GFP (test targeting construct; FIG. 9) in transgenic tobacco by RT-PCR amplification of G4dbd in total RNA extracted from independent tobacco transformed lines, showing the expected bands from independent tobacco transformed lines (individual line number indicated at the top, lane 1 to 14), DNA size Marker (lane M), positive control (lane Pc);

FIG. 20 presents an expression analysis of pKM24cTP-G4dbd-GFP (test targeting construct; FIG. 9) in transgenic tobacco by real time quantitative RT-PCR (qRT-PCR) amplification of G4dbd in independent lines (twelve independent transgenic tobacco cv. Samsun NN, R1 progeny, 2nd generation), presented as a histogram showing the average initial copy number per 100 ng of total RNA in each independent transgenic line, as indicated by number at the bottom (Rt=negative control, Sn=untransformed Samsun NN tobacco);

FIG. 21 presents laser scanning confocal microscope images showing a transient expression analysis of empty vector pKM24 (FIG. 1) after Agro-infiltration into Nicotiana benthamiana leaf tissue, shown by laser scanning confocal microscope imaging, with panel A showing no GFP fluorescence, panel B showing chlorophyll autofluorescence (red color), and panel C providing an overlay image by combining the images of panels A and B;

FIG. 22 presents laser scanning confocal microscope images showing a transient expression analysis of pKM24-GFP (positive control vector; FIG. 3) after Agro-infiltration into N. benthamiana leaf tissue, showing GFP expression in the nucleus (yellow arrow) and throughout the subsidiary cell, with chloroplasts (white arrow) showing red color due to chlorophyll autofluorescence;

FIG. 23 presents laser scanning confocal microscope images showing a transient expression analysis of pKM24cTP-G4dbd (delivery construct; FIG. 10) and pK5uasPrnn-GFP (passenger construct; FIG. 12) after co-Agro-infiltration into N. benthamiana leaf tissue, showing GFP fluorescence localized in the nucleus (yellow arrow; panel A) as well as in a few chloroplasts (white arrow), with panel B showing chlorophyll autofluorescence (red color), and panel C providing an overlay image by combining the images of panels A and B;

FIG. 24 presents laser scanning confocal microscope images showing a second transient expression analysis of pKM24cTP-G4dbd (delivery construct; FIG. 10) and pK5uasPrnn-GFP (passenger construct; FIG. 12) after co-Agro-infiltration into N. benthamiana leaf tissue, showing GFP fluorescence localized in the nucleus (yellow arrow; panel A) as well as in a few chloroplasts (white arrow), with panel B showing chlorophyll autofluorescence (red color), and panel C providing an overlay image by combining the images of panels A and B;

FIG. 25 presents laser scanning confocal microscope images showing a third transient expression analysis of pKM24cTP-G4dbd (delivery construct; FIG. 10) and pK5uasPrnn-GFP (passenger construct; FIG. 12) after co-Agro-infiltration into N. benthamiana leaf tissue, showing GFP fluorescence localized in the nucleus (yellow arrow; panel A) as well as in a few chloroplasts (white arrow), with panel B showing chlorophyll autofluorescence (red color), and panel C providing an overlay image by combining the images of panels A and B;

FIG. 26 presents a transient expression analysis after co-electroporation of pKM24cTP-G4dbd (delivery construct, FIG. 10) and pK5uasPrnn-GFP (passenger construct including a GFP reporter gene; FIG. 12) in tobacco protoplasts; and

FIG. 27 presents a transient expression analysis after co-electroporation of pKM24cTP-G4dbd (delivery construct, FIG. 10) and pK5uasPrnn-LUC (passenger construct including a LUC reporter gene; FIG. 13) in tobacco protoplasts.

### DETAILED DESCRIPTION OF THE ILLUSTRATED EMBODIMENTS

In the following detailed description of the illustrated embodiments, reference is made to the accompanying drawings that form a part hereof and in which are shown, by way of illustration, specific embodiments in which the invention may be practiced. These embodiments are described in sufficient detail to enable those skilled in the art to practice the invention. Also, it is to be understood that other embodiments may be utilized and that process, reagent, software, and/or other changes may be made without departing from the scope of the present invention. Certain patent and non-patent references are cited in the present disclosure, the contents of each of which are incorporated specifically by reference herein unless otherwise indicated.

### EXAMPLE 1

### Strategy for Redirecting Nuclear Targeting DNA Binding Protein Fused with Chloroplast Targeting Sequence into Chloroplasts

A) Transient Expression:

This approach used was to test certain proteins that specifically bind to DNA in order to determine if they could be redirected to the chloroplast. A protein that was selected as a possible candidate was the Gal 4 DNA binding protein domain (G4dbd), a transcriptional activator from yeast that has been shown to have 147 amino acids at the amino terminal region and contain a DNA binding domain that binds to a nucleotide recognition sequence and targets heterologous proteins to the nucleus of animal cells (Forminaya and Wels, 1996; Uherek et al., 1998; Chan et al. 1998). A chloroplast target peptide sequence (cTP; from pea rbcS) was fused in frame to the G4dbd along with a GFP, to visualize localization. The physical map of this construct is shown in FIG. 9.

First, chloroplast transit peptide (cTP), Gal4 DNA binding domain (G4dbd), and green fluorescence protein (GFP) sequence were PCR amplified using appropriately designed PCR primers. The primers used are listed in Table 1.

TABLE 1
Primers used for PCR amplification.

| | | | |
|---|---|---|---|
| SEQ ID NO | Name | Sequence Description | Designed PCR products |

| | | | |
|---|---|---|---|
| 001 | 5′gfpN#883 | 5′-d(CGATTGCCCATGGGTAAAGGA | GFP sequence |
| | | GAACTT)-3′, a 27-mer forward primer | of general structure |
| 002 | 3′gfpS#884 | 5′-d(TGCAGGAGCTCCTAGATAGATCTGT | 5′-NcoI-gfp-Sst1- |
| | | ATAGTTC)-3′, a 32-mer reverse primer | (Seq. ID: 011 |
| 003 | 5′VD2Xb#885 | 5′-d(GCGGGCTCTAGAATGCCCGATC | VirD2 sequence |
| | | GTGCTCAAGTT)-3′, a 33-mer forward | of general structure: |
| | | primer | 5′-XbaI-VirD2-C- |
| 004 | 3′VD2NS#886 | 5′-d(CTTGAGCGGGAGCAGGCTCCCATG | NcoI-SstI-3′, |
| | | GGAGCTCCTGC)-3′, a 35-mer reverse | Seq. ID: 012 |
| | | primer | |
| 005 | 5′cTP(p)X#887 | 5′-d(GCGGCCTCGAGATGGGTTCTATGA | cTP of physical |
| | | TATCCTCTTCC)-3′, a 35-mer forward | structure 5′- |
| | | primer | XhoI-cTP-XbaI- |
| 006 | 3′cTP(p)XbNS | 5′-d(ATGCAGGAGCTCCATGGGTCTAGA | C-NcoI-SstI-3′, |
| | #888 | GCACTTTACTCTTCCACCATT)-3′, a 45- | Seq. ID: 013 |
| | | mer reverse primer | |
| 007 | 5′cTP(p)H#889 | 5′-d(GCGGCAAGCTTATGGGTTCTATGATA | cTP of physical |
| | | TCCTCTTCC)-3′, a 35-mer forward | structure 5′- |
| | | primer | HindIII-cTP-XbaI- |
| 008 | 3′cTP9(p)XbNS | 5′-d(ATGCAGGAGCTCCATGGGTCTAGA | C-NcoI-SstI-3′, |
| | #888 | GCACTTTACTCTTCCACCATT)-3′, a 45- | Seq ID: 014 |
| | | mer reverse primer | |
| 009 | 5′gdbdXb#890 | 5′-d(GCGGCTCTAGAATGAAGCTACTGTC | G4dbp of physical |
| | | TTCT)-3′, a 29-mer forward primer | structure 5′-G4dbd- |
| 010 | 3′gdbdND#891 | 5′-d(GCAGGAGCTCCCATGGGAATCGATAC | C-NcoI-SstI-3′, |
| | | AGTCAACTG)-3′, a 35-mer reverse primer | Seq. ID 015 |
| 018 | 5′epb- | 5′-d(GCGGAATTCCTGCAGGGATCCGCTCCCCCGC | Prnn promoter, |
| | prnn#972 | CGTCGTTC)-3′, a 39-mer forward | physical map: |
| | | primer | 5′-EcoR1-Psti-BamHi- |
| 019 | 3′H-prnn#973 | 5′d(ATGAAGCTTAAATCCCTCCCTACAACT)-3′, | HindIII-3′, |
| | | a reverse primer, a 27-mer reverse | Seq. ID: 024 |
| | | primer | |
| 020 | 5′P-5UAS#988 | 5′-d(GCGGCTTGCATGCCTGCAGGT)-3′, a | 5UAS physical map: |
| | | 21-mer forward primer | 5′-PstI-5UAS-BamHI, |
| 021 | 3′B5uas#989 | 5′d(ATGGGATCCTCTAGCGTCTCCGCTCGG)-3′, | Seq. ID: 025 |
| | | a 27-mer reverse primer | |
| 022 | 5′XB-psA3#976 | 5′-d(GCGTCTAGAGATCCTGGCCTAGTCTA | psbA3′ terminator, |
| | | T)-3′, a 27-mer forward primer | physical map: 5′- |
| 023 | 3′-C1-psA#977 | 5′d(ATGATCGATTCGAATATAGCTCTTCTT)-3′, | XbaI-psbA3′-ClaI- |
| | | a 27-mer reverse primer | 3′, Seq. ID: 026 |

All PCR amplified fragments were cloned into pGEMTeasy (Promega Life Science, Madison, Wis., USA). Appropriate DNA fragments were isolated after restriction with specific restriction enzymes, and were sub-cloned into the corresponding sites of vector pBluescriptII KS⁺. DNA sequencing was done to confirm the sequence integrity of all DNA fragments. The primers used to confirm sequence integrity are set forth in Table 2, and the sequencing results are included herein as SEQ ID NOs: 10-16 and 23-28.

TABLE 2

Gene specific sequencing primers used to confirm
the sequence integrity of constructs

| SEQ | | |
|---|---|---|
| ID NO | Primer Name | Sequence description |
| 030 | 3′Vd2#898 | 5′-d(AATGATAACTTGAGCACGAT)-3′, |
| | | 20-mer antisense sequence |
| 031 | 5′Vd2#899 | 5′-d(AACTTATGACGAAAGTCAGC)-3′, |
| | | 20 mer sense sequence |
| 032 | 3′Vd2#900 | 5′-d(GAGATATCTTCAGCCAGCCG)-3′, |
| | | 20-mer antisense sequence |
| 033 | 5′Vd2#901 | 5′-d(GCGAGCCGACGAGCAGAACG)-3′, |
| | | 20-mer sense sequence |
| 034 | 3′Gfp#902 | 5′d(CTTGACTTCAGCACGTGTCT)-3′, |
| | | 20-mer antisense sequence |
| 035 | 3′G4#903 | 5′-d(GAAAAATCAGTAGAAATAGC)-3′, |
| | | 20-mer antisense sequence. |
| 036 | 5′G4#904 | 5′-d(AAGGCTAGAAAGACTGGAACA)-3′, |
| | | 21-mer sense sequence |

The designed genetic elements were assembled in Bluescript for plasmids pBcTP-G4dbd-GFP. After confirming the sequence integrity of the assembled products, the fragment with general structure 5′-HindIII-cTP-G4dbd-GFP-SstI-3′ was cloned into the corresponding site of plant expression vector pKM24 (Dey and Maiti 1999a, b) to generate plasmid pKM24-TP-G4dbd-GFP (FIG. 9). The plant expression construct was tested in transient expression experiments by bombardment or protoplast electroporation (see below) to show the expression of reporter GFP gene in chloroplasts and nucleus. Expression with a chimeric reporter GFP fusion gene was under the control of a Mirabilis Mosaic Virus (MMV) promoter (Dey and Maiti, 1999a, b) and a rbcS terminator.

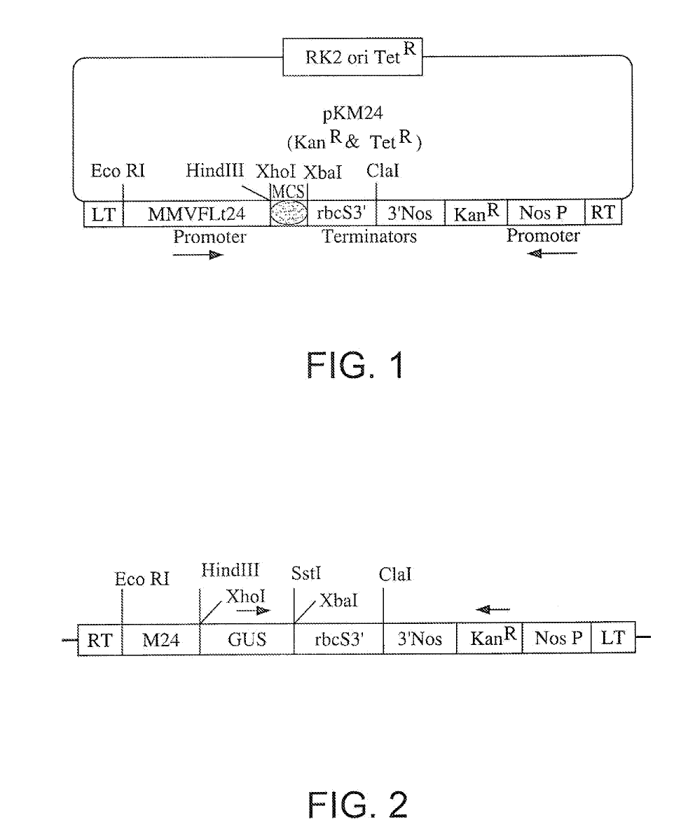

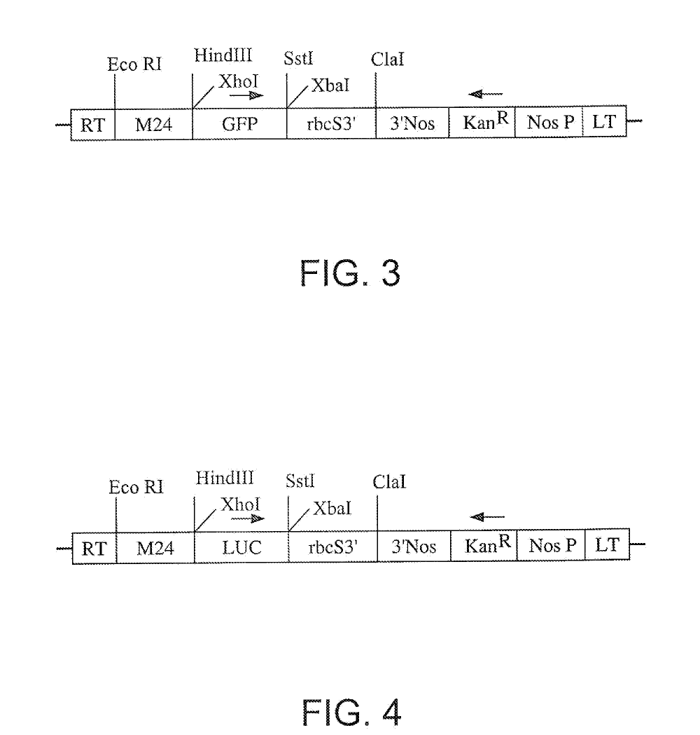

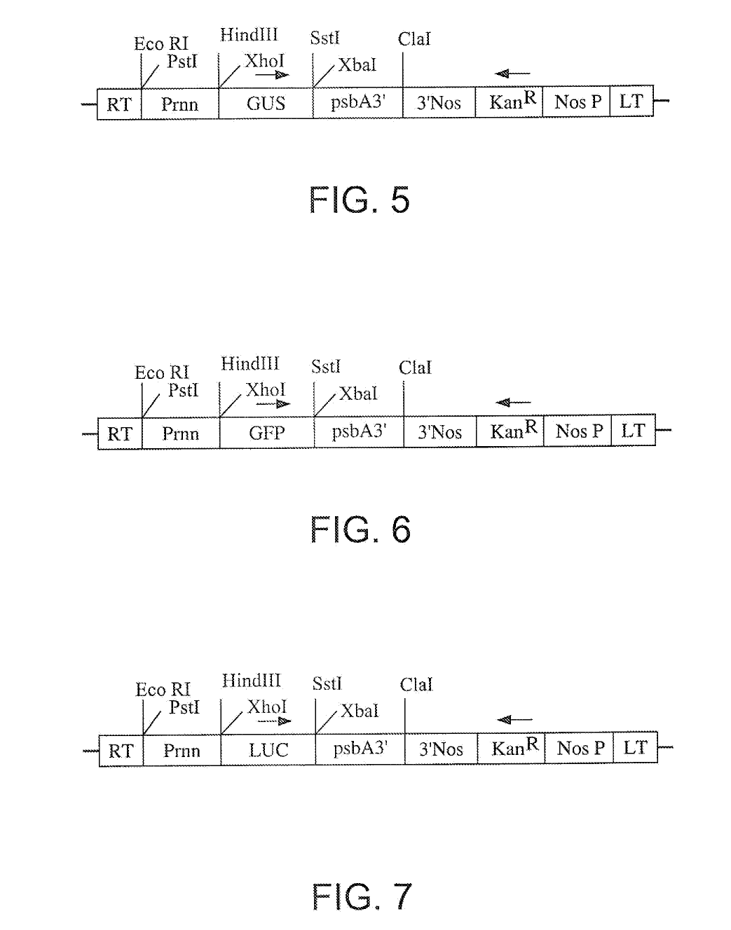

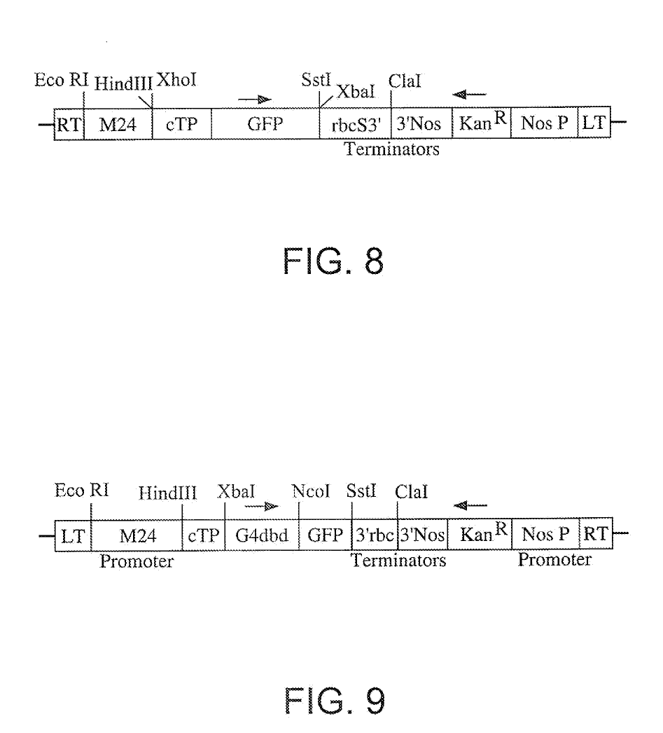

For transient gene expression analysis, the following constructs were used for biolistic/microprojectile bombardment experiments:
a) pKM24 (empty vector used as a negative control, FIG. 1)
b) pKM24-GFP (positive control, FIG. 3)
c) pKM24cTP-GFP (positive chloroplast targeting vector, FIG. 8)
d) pKM24cTP-G4dbd-GFP (Test construct, FIG. 9)

A PDS-1000He device (Sanford et al., 1991) was used for bombardment. Microprojectiles were prepared by mixing 7.5 mg of gold particles/shot (Bio-Rad, Hercules, Calif., USA) with 20 μl (5 μg/μl) plasmid DNA for all constructs. 250 μl of CaCl2 (2.5 mM), and 100 μl spermidine (100 mM). Ten microliters of the suspension were delivered to each macrocarrier and used for each bombardment. Four-week old tobacco leaf discs of Samsun NN & Kentucky 160 were used. A pressure of 1400 kPa and distance ranging from 12.5 to 17.5 cm with pulse duration of 50 m-sec were used.

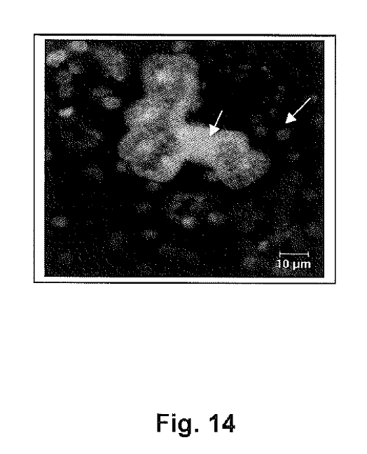

The bombarded leaf discs were incubated for 48 hours on TOM medium in the dark. Transient expression of GFP in the chloroplast was assayed 12 hours after incubation using a laser scanning confocal microscope (Model-Leica TCSNT). The nucleus was also examined for fluorescence, to determine if the addition of a target sequence abolished any nuclear localization. For each construct, the GFP fluorescence, chlorophyll autofluorescence and the overlay of the two are shown either in a single guard cell or in the subsidiary cells. As expected when GFP is expressed alone, it is found throughout the cytosol and in the nucleoplasm, but is excluded from the chloroplast (FIG. 14).

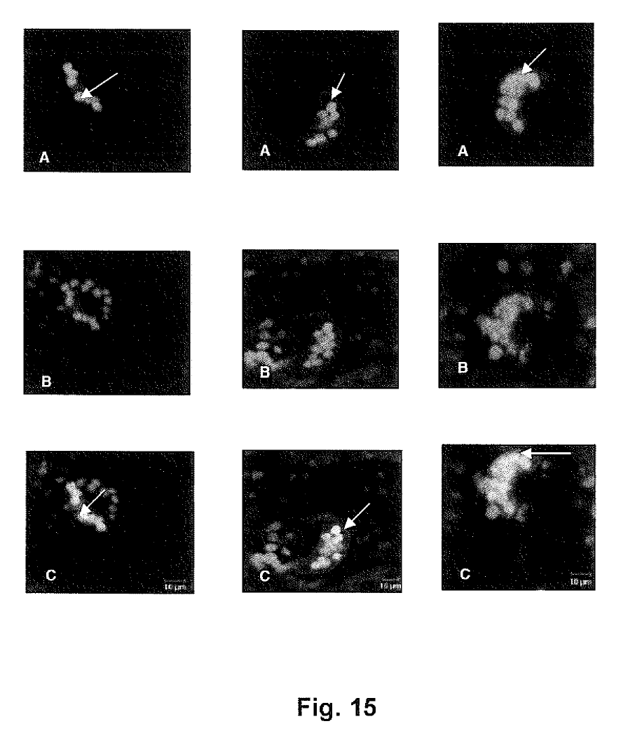

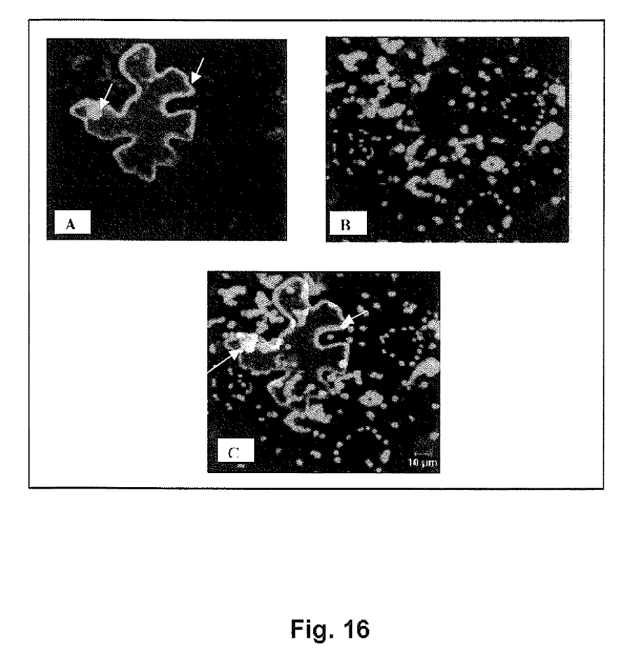

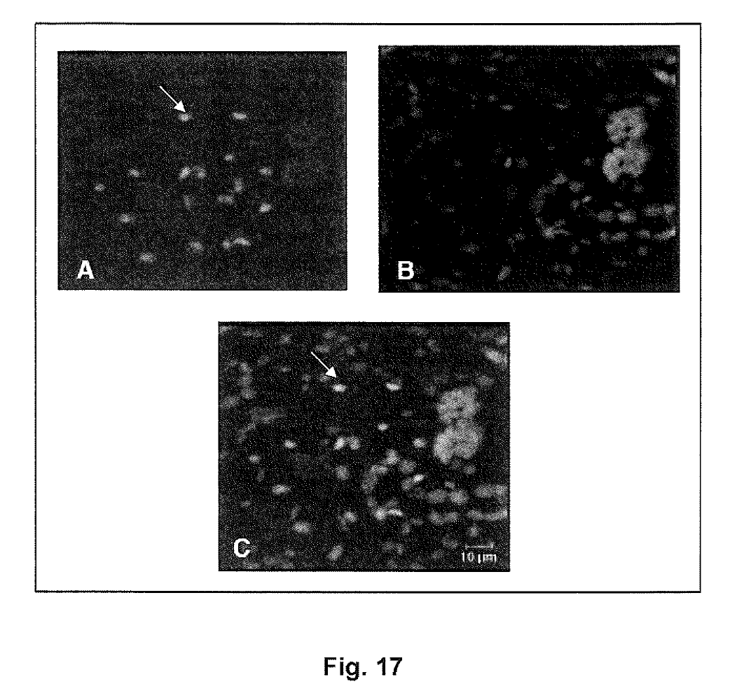

In contrast, GFP expressed as a fusion protein with the chloroplast transit peptide is found exclusively in the chloroplasts, as evidenced by the exact superimposition of GFP and chlorophyll fluorescence in the overlay (FIG. 15). Similarly, GFP expressed as a fusion protein with Gal4dbd and the chloroplast transit peptide shows that GFP is localized both in the chloroplasts as well as the nucleus (FIG. 16 and FIG. 17), respectively. These results demonstrate successful redirection of GFP using a chloroplast-targeting sequence fused with Gal4dbd.

B) Stable Expression:

Stable expression studies were earned out using the construct pKM24cTp-Gal4dbd-GFP (FIG. 9) by transforming Nicotiana tabacum cv. Samsun NN through Agrobacterium mediated transformation using the leaf disc co-cultivation method as described previously (Maiti et al., 1993). Plants were rooted in kanamycin selection medium and transferred to the green house for further hardening prior to collection of seeds from ten different independent lines.

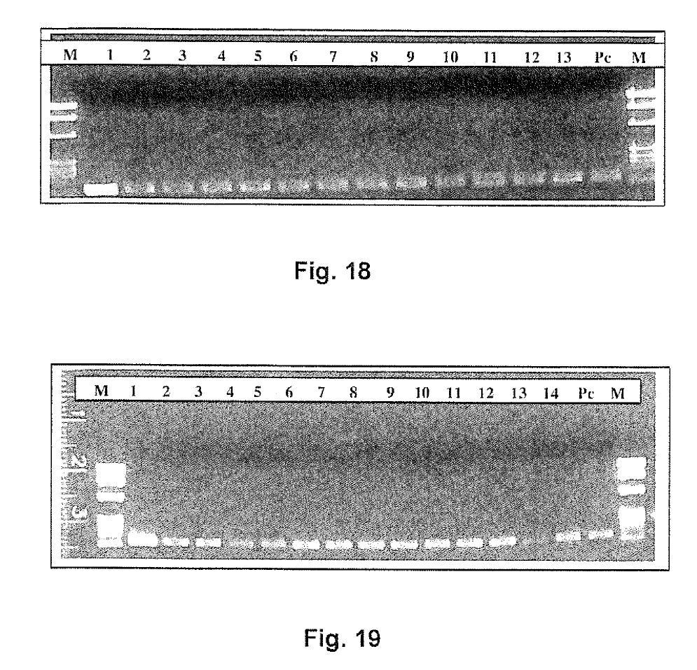

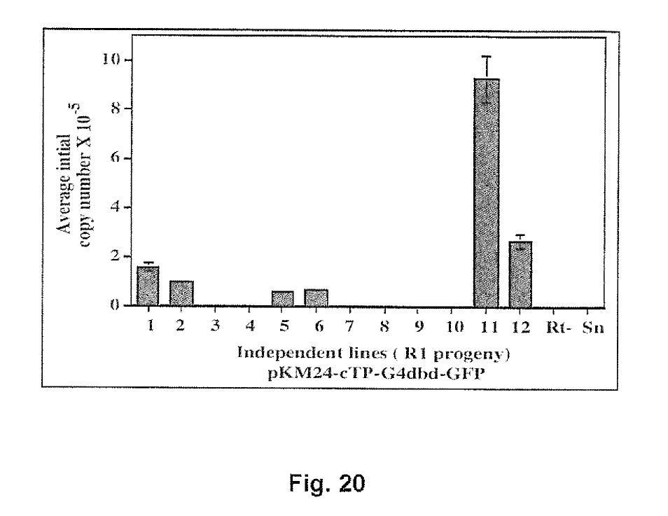

Segregation analysis of Kan^{R} marker gene was performed for the different independent transgenic lines. Molecular analysis for the Gal4dbd fragment was conducted by PCR of genomic DNA (FIG. 18), RT PCR (FIG. 19) and qRT PCR (FIG. 20) of transgenic lines (R I lines, KanR). The primers used are listed in Table 3.

TABLE 3

Gene specific primers used for molecular analysis
(PCR, RT-PCR, qRT-PCR) of gene expression

| SEQ | | |
|---|---|---|
| ID NO | Primer Name | Sequence description |
| 037 | 5′cTP#32 | 5′-d(GGTTCTATGATATCCTCTTC)-3′, |
| | | 20-mer sense sequence |
| 038 | 3′cTP#933 | 5′-d(TCTAGAGCACTTTACTCTTC)-3′, |
| | | 20-mer anti sense sequence |
| 039 | 5′VD2#934 | 5′-d(ATGCCCGATCGTGCTCAAGT)-3′, |
| | | 20-mer sense sequence |
| 040 | 3′VD2#935 | 5′-CCCTGCCTGCTCCCGCTCAA)-3′, |
| | | 20-mer antisense sequence |
| 041 | 5′GFP#936 | 5′-d(ATGGGTAAAGGAGAACTTTT)-3′, |
| | | 20-mer sense sequence |
| 042 | 3′GFP#937 | 5′-d(CTAGATAGATCTGTATAGTT)-3′, |
| | | 20-mer antisense sequence |
| 043 | 5′G4dbd#938 | 5′-d(ATGAAGCTACTGTCTTCTAT)-3′, |
| | | 20-mer sense sequence |
| 044 | 3′G4dbd#939 | 5′-d(GGGAATCGATACAGTCAACT)-3′, |
| | | 20-mer antisense sequence |

The analysis showed the integration and expression of the transgene in independent lines. Leaves of four-week old tobacco lines examined by confocal microscope showed the GFP expression both in chloroplasts and nucleus. Therefore, the experiments demonstrated a successful redirection of GFP using chloroplast-targeting sequence fused with Gal4dbd in transgenic plants.

### EXAMPLE 2

### Co-Agro-Infiltration Analysis of the Delivery Construct M24cTP-G4dbd and the Passenger Construct 5Xuas-Prnn-GFP-psb3′

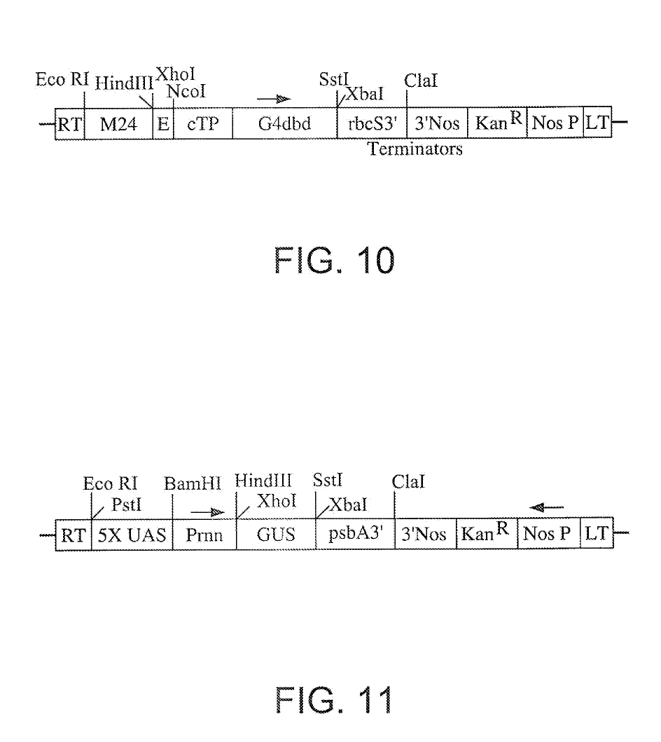

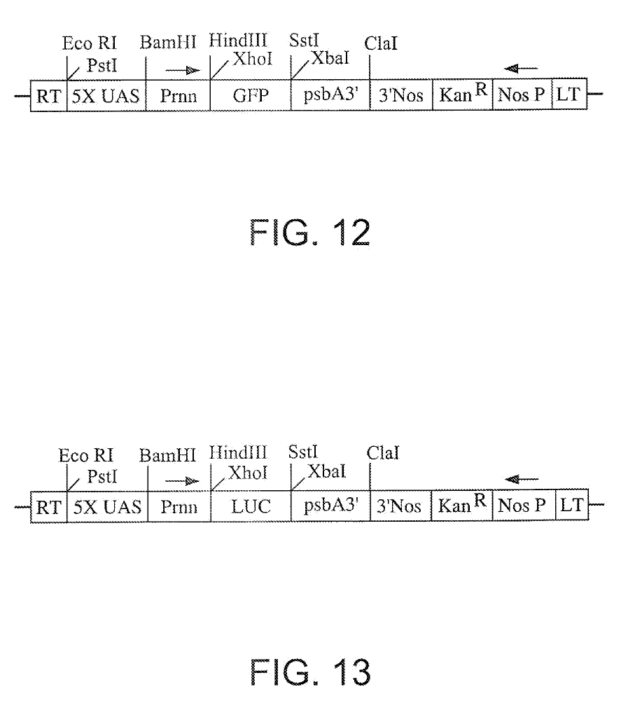

The experiments described herein were performed to show transient expression of GFP in the chloroplast using delivery and passenger constructs according to the present disclosure. For Agro-infiltration and Co-Agro-infiltration studies, all constructs were first cloned into the binary vector pKM24 (FIG. 1), and were mobilized into Agrobacterium strain GV3850 as described below. The following constructs were used:
a) pKM24 (empty vector used as a negative control, FIG. 1),
b) pKM24-GFP (a positive control, FIG. 3),
c) pKM24cTP-G4dbd (delivery construct, FIG. 10), and
d) pK5uas-Prrn-GFP-psbA3′ (passenger construct, FIG. 11).

Agrobacterium strain GV3850 harboring the constructs were grown on plates containing rifampicin (100 ug/ml), tetracycline (15 μg/ml) and kanamycin (100 μg/ml). The cells were scraped and mixed in 4 ml of 10 mM MES and 10 mm MgCl, to attain an OD of 0.6 at λ600. Acetosyringone was added at a concentration of 0.1M and the mixture was kept on the table for three hours at room temperature. A syringe without a needle was used to infiltrate the cell suspension from the ventral side of the leaf of potted plants of Nicotiana benthamiana. The edge of the infiltrated area was marked and the plants were kept in a growth room under defined conditions of temperature and light for 48 hours prior to observation under a scanning laser confocal microscope.

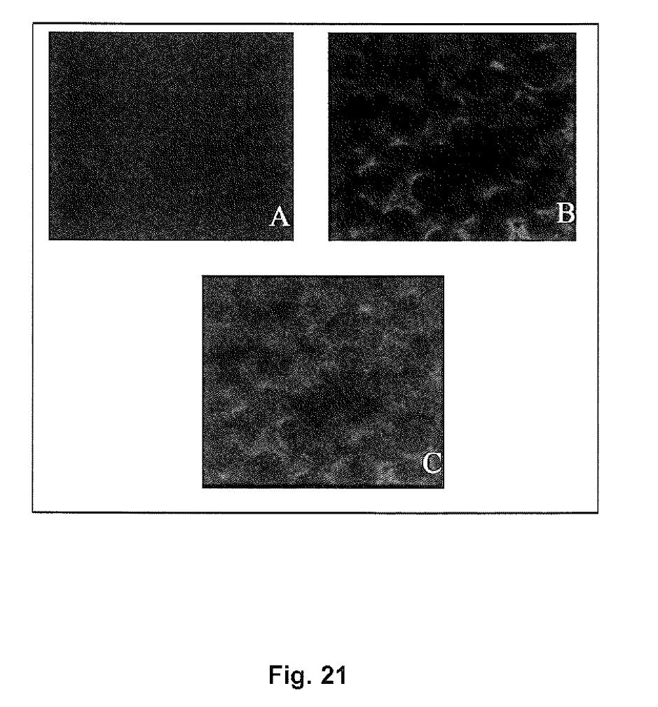

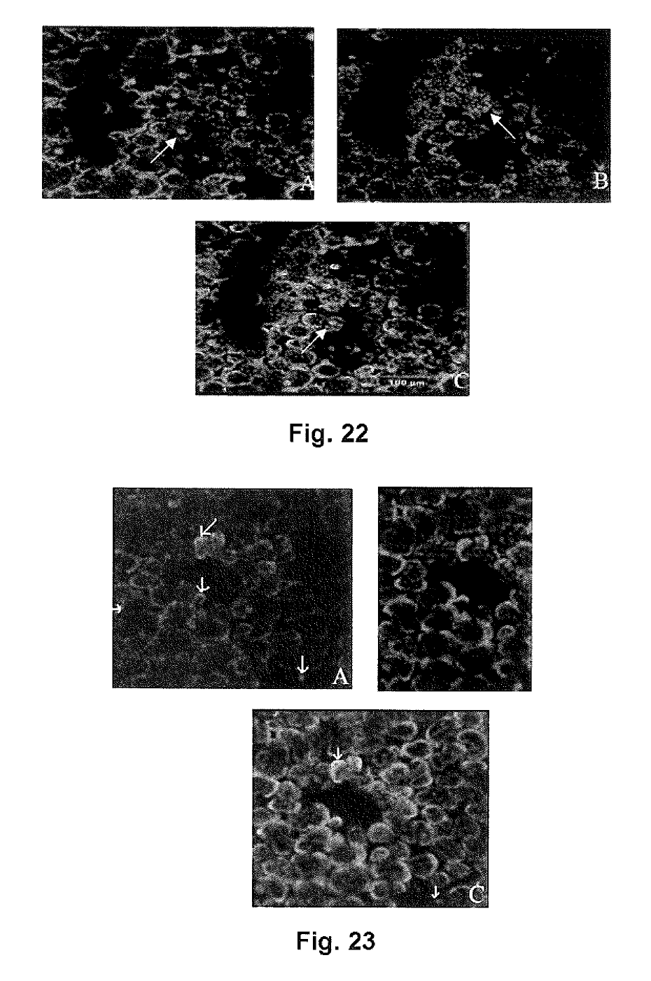

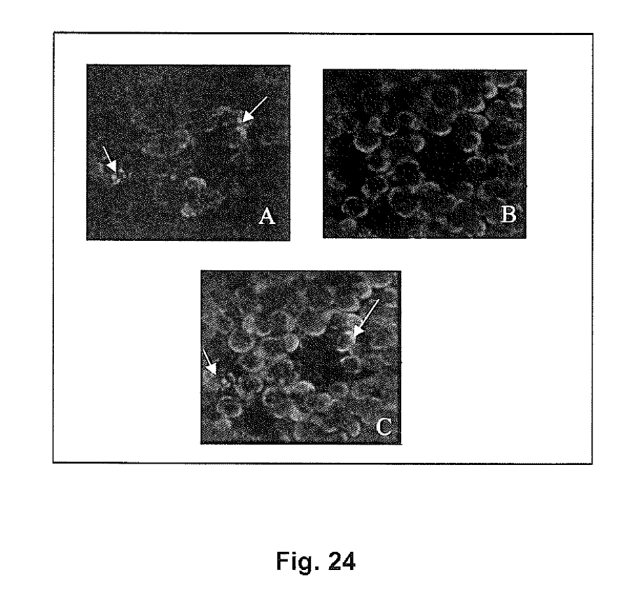

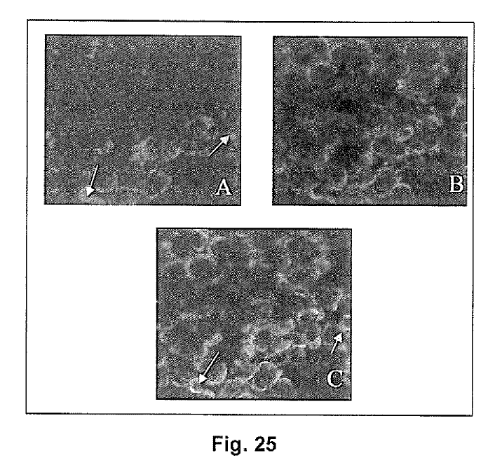

The transient expression analysis upon co-Agro-infiltration of the constructs pKM24cTP-G4dbd and 5XUAS-Prnn-GFP-psbA3′ showed that apart from GFP expression mostly in the nucleus, there were also a few chloroplasts in which GFP was detected within the guard cells of the chloroplast (FIGS. 23, 24, and 25). The frequency of chloroplasts expressing GFP was >1%. A positive control (pKM24-GFP) and a negative control (empty vector pKM24) were also infiltrated. FIG. 21 shows the image of a negative control, which shows no GFP expression. The positive control using pKM24-GFP showed GFP expression throughout the cytoplasm and the nucleus excluding the chloroplasts (FIG. 22). These results of co-Agro-infiltration studies clearly demonstrate that the delivery constructs as described herein are capable of transferring the passenger constructs into the chloroplasts, and that the foreign DNA so transferred is expressed.

### EXAMPLE 3

### Relative Level of Expression Analysis Upon Co-Electroporation of the Delivery Construct pKM24-cTP-G4dbd and the Passenger Constructs pK5uas-Prnn-GFP-psbA3, pK5uas-Prnn-LUC-psbA3 in Tobacco Protoplasts

Experiments were performed to show transient expression of co-electroporated constructs in protoplasts using GFP and LUC as reporter genes. Protoplasts from three-day old cell suspension cultures of Tobacco (xanthii-brad) were isolated as described in detail previously (Dey and Maiti 1999a). Constructs as described below were electroporated using a Bio-Rad Gene Pulser II set to 200 V and 950 μF as described earlier (Dey and Maiti, 1999a).

For experiments using LUC as a reporter gene, the following constructs were assayed:
a) pKM24 (empty vector used as a negative control. FIG. 1),
b) pKM24-LUC-rbcs3′ (a positive control, FIG. 4), and
c) pKPrnn-LUC-psbA3′ ( a control vector, FIG. 7).

For co-electroporation experiments, the following constructs were used:
a) pKM24cTP-G4dbd (Delivery construct, FIG. 10), and
b) pK5uas-Prrn-LUC-psbA3′ (Passenger construct, FIG. 13)
   All constructs were assayed for luciferase activity after 24 hours of incubation using a Luminometer (Tuner systems).

Similarly for experiments using GFP as a reporter gene, the following constructs were assayed,
a) pKM24 (empty vector, a negative control, FIG. 1),
b) pKM24-GFP-rbcs3′ (a positive control, FIG. 3), and
c) pKPrnn-GFP-psbA3′ (a control vector, FIG. 6).

For co-electroporation experiments, the following constructs were used:
a) pKM24cTP-G4dbd (delivery construct, FIG. 10), and
b) pK5uas-Prnn-GFP-psbA3′ (passenger construct, FIG. 11).

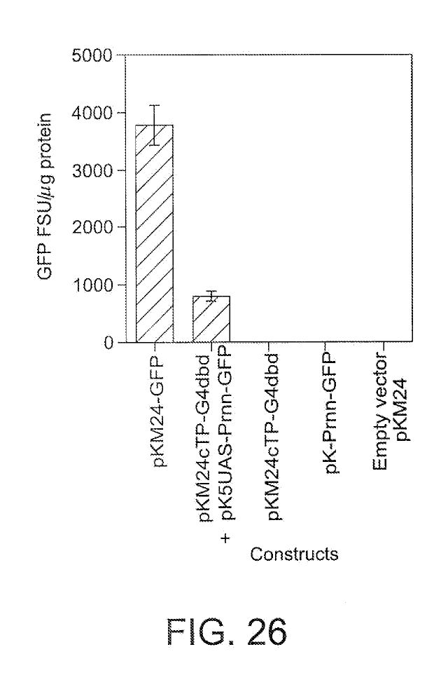

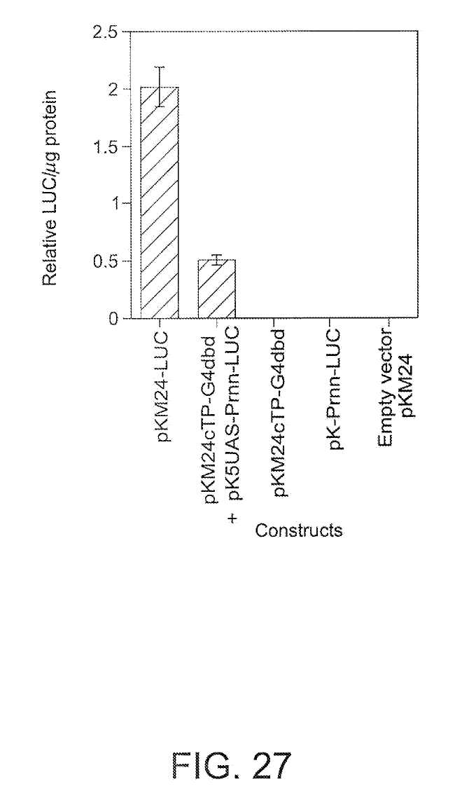

All constructs were assayed for GFP activity after 24 hours of incubation using a Fluorometer (Tuner systems). Co-electroporation of the delivery/passenger constructs incorporating GFP and LUC reporter genes (pKM24cTP-G4dbd/5XUAS-Prrn-GFPpsbA3′ and pKM24cTP-G4dbd/5XUAS-Prnn-LUC-psbA3′, respectively), along with positive and negative controls was done in tobacco protoplasts. Both positive control constructs pKM24-GFP and pKM24-LUC showed GFP activity (FIG. 26) and LUC activity (FIG. 27). No LUC or GFP activity was shown from the constructs pK-Prnn-LUC or pK-Prnn-GFP, which was expected as the Prnn promoter is active only in the plastid. Empty vector without any reporter gene (negative control; FIG. 1) showed no activity. Co-electroporation of the delivery construct pKM24cTP-G4dbd and passenger constructs either with GFP or LUC reporter gene showed activity. These results of co-electroporation studies in tobacco protoplasts clearly demonstrate that the delivery construct described herein is capable of transferring the passenger construct into the chloroplasts, and that the foreign DNA so transferred is expressed.

Competent transgenic plants developed with a specific delivery construct will be useful for transforming chloroplast in a way that will be very customer friendly.

One of ordinary skill in the art will recognize that additional embodiments of the invention are also possible without departing from the teachings herein. This detailed description, and particularly the specific details of the exemplary embodiments, is given primarily for clarity of understanding, and no unnecessary limitations are to be imported, for modifications will become obvious to those skilled in the art upon reading this disclosure and may be made without departing from the spirit or scope of the invention. Relatively apparent modifications, of course, include combining the various features of one or more figures with the features of one or more of other figures.

### REFERENCES

Benfey P N, Chua N H (1989) Regulated genes in transgenic plants. Science 244: 174-181.
Boyer C T, Shannon J C, Hardison R C (eds.) Physiology, Biochemistry, and Genetics of nongreen Plastids. (Amer. Soc. Plant Physiol., Rockville, Md. 1989)
Chan C K, Hubner S, Hu W, Jans D A (1998) Mutual exclusivity of DNA binding and nuclear localization signal recognition by the yeast transcription factor Gal4: implications for nonviral DNA delivery, Gene Therapy 5: 1204-1212.
Daniell H, Datta R, Varma S, Gary S, and Lee S B (1998) Containment of herbicide resistance through genetic engineering of the chloroplast genome. Nat Biotechnol 16: 346-348.
Daniell H, McFadden B A (1987) Uptake and expression of bacterial and cyanobacterial genes by isolated cucumber etioplasts. Proc. Natl. Acad. Sci. USA 84: 6349-6353
Daniell H. Vivekananda J, Nielson B, Ye G, Tewari K K, Sanford J C (1990) Transient foreign gene expression in chloroplasts of culture tobacco cells following biolistic delivery of chloroplast vectors. Proc Natl Acad Sci USA 87: 88-92.
De Cosa B, Moar W. Lee S B, Miller M, Daniell H (2001) Overexpression of the Bt cry2Aa2 operon in chloroplasts leads to formation of insecticidal crystals. Nat. Biotechnol 19: 71-74.
Dey N, Maiti I B (1999b). Further characterization and expression analysis of mirabilis mosaic virus (MMV) full-length transcript promoter with single and double enhancer domains in transgenic plants. Transgenics 3, 61-70.
Dey N, Maiti I B (1999a). Structure and promoter/leader deletion analysis of mirabilis mosaic virus (MMV) full-length transcript promoter in transgenic plants. Plant Mol. Biol 40: 771-782.
Fromm M E, Taylor L P, Walbot V (1986) Stable transformation of maize after gene transfer by electroporation. Nature 319: 791-793.
Gruissem W (1989) Chloroplast gene expression: How plants turn their plastids on. Cell 56: 161-170.
Jansen R K, Raubeson L A, Boore J L, dePamphilis C W, Chumley T W, Haberle R C, Wyman S K, Alverson A J, Peery R, Herman S J, Fourcade H M, Kuehl J V, McNeal J R, LeebensMack J, Cui L (2005) Methods for obtaining, and analyzing chloroplast genome sequence. Methods in Enzymology 395: 348-384.
Klein T M, Gradzil T, Fromm M E, Sanford J C (1988) Factors influencing gene delivery into Zea inays cells by high velocity microprojectiles. Bio/Technology 6: 559-563.
Krens F A, Molendijk L, Wullems G J, Schilperoort R A (1982) In vitro transformation of plant protoplast with Ti-plasmid DNA. Nature 296: 72-74.
Lee S-B, Kaittanis C, Hostetler J, Town C, Jansen R K, Daniell H (2006) The complete chloroplast genome sequence of Gossypiuin hirsutuin: organization and phylogenetic relationships to other angiosperms. BMC Genomics 7: 61-98
Maiti I B and Hunt A (1992)—Developing Genetically Engineered Disease, Pest and Herbicide Resistance in Tobacco, 46th Tobacco Chemists' Research Conference, Recent advances in tobacco Sciences, Highlights of Current Research on Tobacco Chemistry, Proceedings of a Symposium at the 46th Meeting of the Tobacco Chemists' Research Conference, vol 18, September 27 30. 1992, pp. 45-68
Maiti I B, Murphy J F, Shaw J G, and Hunt A G (1993) Plants that express a potyvirus proteinase gene are resistant to virus infection. Proc. Natl. Acad. Sci. USA. 90: 6110-6114.
Palmer J D (1985) Comparative organization of chloroplasts genomes. Ann Rev Genet 19: 325354.
Polans N 0, Corriveau J L, Coleman A W (1990) Plastid inheritance in Pisum sativum L. Curr. Genet. 18: 477-480.
Shinozaki K, et al., (1986) The complete nucleotide sequence of the tobacco chloroplast genome; its organization and expression. EMBO J 5: 2043-2049.
Wagner G (1992) “Improving Tobacco Through Metabolic Engineering: Promise and Obstacles”, 46th Tobacco Chemists' Research Conference, Recent advances in tobacco Sciences, Highlights of Current Research on Tobacco Chemistry, Proceeding of a Symposium at the 46th Meeting of the Tobacco Chemists' Research Conference, vol 18, Sep. 27-30, 1992, pp. 45-68
Weising K, Schell J, Kashi G (1988) Foreign genes in plants: transfer structure, expression and applications. Annu Rev Genet 22: 421-477.
Zurawski G, Clegg M T (1987) Evolution of higher-plant chloroplast DNA-encoded genes: implication for structure-function and phylogenetic studies. Ann. Rev. Plant Physiol. 38: 391418.

What is claimed is:

1. A method for plastid transformation of a plant cell, comprising:
providing a delivery construct including a nucleotide sequence encoding at least one fusion protein comprising a chloroplast transit peptide and a DNA binding domain, expression of said at least one fusion protein nucleotide sequence being controlled by a promoter;
providing a passenger construct including a nucleotide sequence encoding at least one foreign protein of interest or fragment thereof desired to be expressed in the plant cell; and
introducing the delivery construct and the passenger construct into the plant cell under conditions whereby the delivery construct transfers the passenger construct into the plant cell plastid and the at least one foreign protein or fragment thereof is expressed in the plant cell.

2. The method of claim 1, including deriving the chloroplast transit peptide from pea rbcS.

3. The method of claim 1, including providing the DNA binding domain as Gal4 DNA binding domain (G4dbd) derived from yeast.

4. The method of claim 1, including providing the delivery construct including a Mirabilis Mosaic virus promoter M24.

5. The method of claim 1, including providing a passenger construct comprising:
a plastid-specific promoter;
at least one nucleotide sequence encoding a protein of interest or fragment thereof under the control of said plastid-specific promoter; and
a terminator sequence.

6. The method of claim 5, including providing a passenger construct including the plastid-specific promoter Prrn.

7. The method of claim 5, including providing a passenger construct including the terminator sequence psbA3′.

8. A delivery system for plastid transformation of a plant cell, comprising:
a delivery construct including a nucleotide sequence encoding at least one fusion protein comprising a chloroplast transit peptide and a DNA binding domain, expression of said at least one fusion protein nucleotide sequence being controlled by a promoter; and
a passenger construct including a nucleotide sequence encoding at least one foreign protein of interest or fragment thereof desired to be expressed in the plant cell.

9. The delivery system of claim 8, wherein the chloroplast-targeting protein is derived from pea rbcS.

10. The delivery system of claim 8, wherein the DNA binding domain is Gal4 DNA binding domain (G4dbd) derived from yeast.

11. The delivery system of claim 8, wherein the delivery construct promoter is Mirabilis Mosaic virus promoter M24.

12. The delivery system of claim 8, wherein the delivery construct is PKM24-cTP-G4dbd.

13. The delivery system of claim 8, wherein the passenger construct comprises:
a plastid-specific promoter;
at least one nucleotide sequence encoding a protein of interest or fragment thereof under the control of said plastid-specific promoter; and
a terminator sequence.

14. The delivery system of claim 13, wherein the plastid-specific promoter sequence is Prrn.

15. The delivery system of claim 13, wherein the terminator sequence is psbA3′.

16. A transgenic plant cell plastid-transformed with the delivery system of claim 8 to express a foreign DNA of interest.

17. A transgenic multicellular plant plastid-transformed with the delivery system of claim 8 to express a foreign DNA of interest.

18. The transgenic multicellular plant of claim 17, wherein the plant is a tobacco plant.
